(19) United States
(12) Patent Application Publication
(10) Pub. No.: US 2022/0304575 A1
(43) Pub. Date: Sep. 29, 2022

(54) Mobile-Powered Desktop ECG System

(71) Applicant: Tricog Health Pte Ltd, Singapore (SG)

(72) Inventors: Zainul Charbiwala, Bengaluru (IN); Vamshi Krishna, Bengaluru (IN); Hamad Mohsin Jowher, Hyderabad (IN); Charit Bhograj, Bengaluru (IN); Udayan Dasgupta, Bengaluru (IN); Abhinav Gujjar, Bengaluru (IN); Achuth Pv, Calicut (IN); Ajay Singh Dahiya, Bengaluru (IN); Shekhar Kumar Gupta, Bengaluru (IN); Yureka Rajendran, Tiruppur (IN)

(21) Appl. No.: 17/638,999

(22) PCT Filed: Aug. 27, 2020
(86) PCT No.: PCT/SG2020/050501

§ 371 Date: Feb. 28, 2022

(30) Foreign Application Priority Data
Aug. 29, 2019 (IN) 201941034744

(51) Int. Cl. A61B 5/00; A61B 5/11; G16H 10/60; G16H 80/00; G16H 40/67

(52) U.S. Cl. CPC A61B 5/0022; A61B 5/0006; A61B 5/743; A61B 5/7475; A61B 5/7264; A61B 5/1112; A61B 5/747; G16H 10/60; G16H 80/00; G16H 40/67

(57) ABSTRACT

The invention provides a system and method for a mobile-powered desktop ECG system configured for remote monitoring of a patient. Analog ECG waveforms from probes attached to a patient are shared with a base ECG unit. The received analog ECG waveforms are converted into digital ECG waveforms and shared with a smartphone that powers the base ECG unit. The digital ECG waveforms are processed in the smartphone through an ECG application and sent to a cloud. The ECG application enables the smartphone connect with a medical practitioner in case a medical emergency is detected.

### CROSS-REFERENCE TO RELATED APPLICATIONS

This application is the United States national phase of International Application No. PCT/SG2020/050501 filed Aug. 27, 2020, and claims priority to Indian Patent Application No. 201941034744 filed Aug. 29, 2019, the disclosures of which are hereby incorporated by reference in their entirety.

### BACKGROUND OF THE INVENTION

### Field of Invention

The present invention relates to a system and method for real-time detection of cardiac abnormalities. More specifically, the invention relates to a mobile powered cloud-connected ECG system for displaying ECG waveforms and receiving a remote diagnosis.

### Description of Related Art

Heart diseases have become one of the major causes of death around the world. Electrocardiograms detect heart rhythms and graphically represent electrical activity of the heart. However, there are requirements for a system to conveniently and accurately determine a patient's cardiac condition in order to detect a health emergency.

In conventional methods, portable health monitoring systems include an ECG capturing device, and transmit ECG information to an application or a medical practitioner. However, such devices comprise certain challenges such as issues in cost, portability, identification of location co-ordinates, signal co-relation, convenience, and comfort of the patient.

Additionally, conventional portable health monitoring systems use a battery, which increases the cost of the device and requires regular usage, repair and maintenance. Additionally, battery-based devices require a charging mechanism, which increases the cost, bulk and weight of the device, thus making it less convenient and affordable.

Thus, in light of the foregoing examination, there is a long-felt need to have a low-cost, portable and adaptable system and method for detecting real-time cardiac abnormalities without a battery, with remote monitoring of the same by a medical practitioner and identification of location co-ordinates during any detected emergency situations.

### SUMMARY OF INVENTION

The principal object of the invention to provide a base ECG unit for displaying ECG waveforms.

It is still another object of the invention to provide a base ECG unit with a QWERTY keyboard for entering various information including patient details.

It is another object of the invention is to provide a system and method for detecting cardiac abnormalities in real-time.

It is another object of the invention to provide a low-cost, portable, off-the-shelf ECG system that can be powered by and used with any mobile phone.

It is another object of the invention to provide a base ECG unit with cloud connectivity for sharing ECG waveforms for feedback.

It is still another object of the invention to provide a display for displaying ECG waveforms and real-time feedback messages from a medical practitioner to a remote operator.

It is another object of the invention to provide an off-the-shelf ECG system that can be configured to use any mobile phone's processor for displaying and analyzing an ECG signal.

It is another object of the invention to provide a speaker for providing audio cues/information to the remote operator.

It is still another object of the invention to provide video-conferencing between the remote operator and the medical practitioner.

It is still another object of the invention to provide a screen-casting feature for enabling a remote medical practitioner to trigger an ECG capture.

It is a further object of the invention to provide a GPS module for collecting location co-ordinates of the patient for identifying optimum traffic routes during any detected critical conditions.

### BRIEF DESCRIPTION OF THE DRAWINGS

This invention is illustrated in the accompanying drawings, throughout which, like reference letters indicate corresponding parts in the various figures.

The embodiments herein will be better understood from the following description with reference to the drawings, in which:

FIG. 1 depicts a system comprising an ECG system, in accordance with an embodiment of the invention.

FIG. 2 depicts a system for identifying real-time ECG waveforms and receiving feedback from the medical practitioner, in accordance with an embodiment of the invention.

FIG. 3 depicts the components of the ECG system, in accordance with an embodiment of the invention.

FIG. 4 depicts a flow chart illustrating a method for using a cloud-connected ECG system, in accordance with an embodiment of the invention.

FIG. 5 depicts a flow chart illustrating a method of real-time remote diagnosis, in accordance with an embodiment of the invention.

### STATEMENT OF INVENTION

The present invention discloses a system and method for displaying an ECG signal of a patient. The present invention further discloses detecting real-time cardiac abnormalities by enabling remote monitoring of the ECG signal by a medical practitioner, and receiving feedback from the medical practitioner.

The system comprises a base ECG unit that connects to a mobile phone. The mobile phone provides power to enable one or more functions of the base ECG unit. The base ECG unit comprises electronic circuits to convert the analog ECG signal from the probes into a digital ECG signal. The base ECG unit further comprises a keyboard, such as a QWERTY keyboard, as well as dedicated specialized keys, in order to enter one or more information including patent information into the base unit, which is reflected on the mobile phone and shared on a server.

The mobile phone can be embedded with algorithms for analyzing the ECG signal, and share information with the server. The digital ECG waveforms are processed through an application in the mobile phone and sent to the server using cloud communication. The algorithm enables the mobile phone to make a connection with the medical practitioner, in case any diagnosis or feedback is required. Similarly, the medical practitioner sends back final diagnosis, feedback and suggested actions to a remote operator of the base ECG unit through the mobile phone. The feedback can be displayed to the remote operator through the display in the mobile phone.

Further, a speaker embedded within the mobile phone can provide audio cues/information to the remote operator, to help them in operating the base ECG unit.

Additionally, during medical emergencies, a current traffic condition of the location of a patient is obtained by collecting location co-ordinates through an in-built GPS module in the mobile phone.

### DETAILED DESCRIPTION OF INVENTION

The embodiments herein and the various features and advantageous details thereof are explained more fully with reference to the non-limiting embodiments that are illustrated in the accompanying drawings and/or detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The embodiments herein below provide the details of a system and method for detecting real-time cardiac signals by receiving ECG waveforms from probes connected to a patient's body and remotely analyzing the same by a medical practitioner. A cloud-connected desktop ECG machine (hereinafter referred to as the base ECG unit) receives analog ECG waveforms from the probes connected to a patient. The analog ECG waveforms are converted into digital ECG waveforms by the base ECG unit. The base ECG unit is connected to a mobile phone, wherein the mobile phone provides power to the base ECG unit. The digital ECG waveforms are further processed by an application in the mobile phone and the processed ECG waveforms are displayed to an operator through the display in the mobile phone.

In an embodiment, specific algorithms of the application incorporated in the mobile phone enable the transmission of processed digital ECG waveforms to a server through cloud communication/computing.

The mobile phone enables communication between the medical practitioner and the remote operator of the base ECG unit, wherein feedback/diagnosis from the medical practitioner is received through the mobile phone and is displayed to the remote operator through the display in the mobile phone. The mobile phone comprises a QWERTY keyboard for providing details of the patient, a speaker for providing cues/information to the operator, and a memory unit for storing ECG data that is not transmitted to the cloud. The QWERTY keyboard facilitates better workflow as compared to other cell phone keyboards.

In an embodiment, video-conferencing is enabled between the operator and the medical practitioner through the mobile phone, and connection to other medical devices/IoT devices is enabled to provide overall health screening for the patient. The mobile phone comprises an in-built GPS module that collects location co-ordinates of the patient's place to identify optimum traffic routes in case of any emergencies/critical condition detected from the captured ECG waveforms.

Referring now to the drawings, where similar reference characters denote corresponding features consistently throughout the figures, there are shown preferred embodiments.

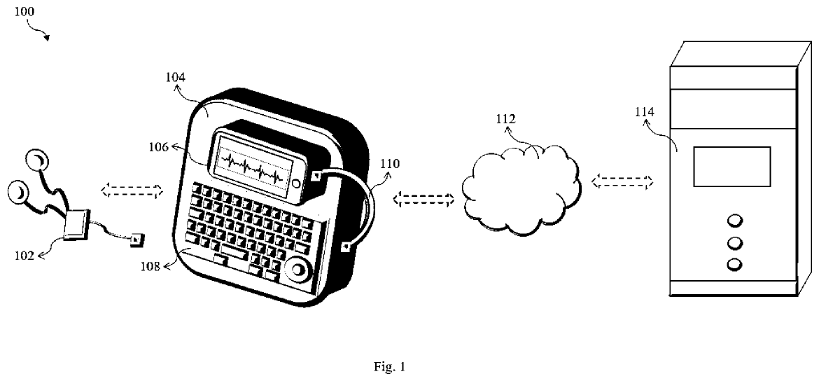

FIG. 1 depicts a system 100 illustrating an ECG base unit 104, in accordance with an embodiment of the invention.

Electronic probes 102 are connected to a patient to detect ECG waveforms of the patient's heart. The electronic probes 102 are connected to a base ECG unit 104. The base ECG unit 104 is connected to a smartphone 106. Further, the base ECG unit 104 comprises ECG processing unit (not shown in the figure), and a keyboard 108.

In an embodiment, the smartphone 106 provides power to the base ECG unit 104 through a wired connection unit 110 between the smartphone 106 and the base ECG unit 104. In an alternate embodiment, the base ECG unit 104 can be powered by a battery or other power sources.

In an embodiment, an operator (not shown in the figure) operates the base ECG unit 104 while reading a patient's ECG waveforms. The operator may use the keyboard 108 to enter one or more patient information into an application installed in the smartphone 106. The patient information can be used to log in to the patient's account, and can be accessed and stored by one or more servers 114 that communicate with the smartphone 106.

In an embodiment, the base ECG unit 104 receives analog ECG waveforms and converts them into digital ECG waveforms. The base ECG unit 104 also transmits the digital ECG waveforms to the smartphone 106.

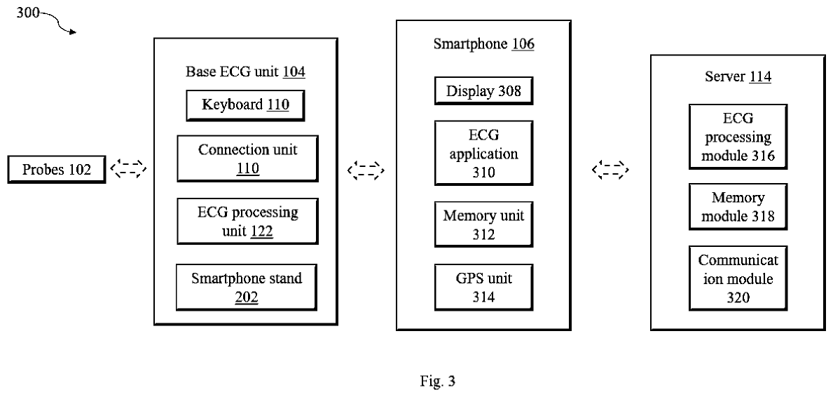

In an embodiment, the ECG waveforms are processed in the smartphone 106 and the processed ECG waveforms are shared with a cloud 112. Further, the smartphone 106 shares the processed ECG waveforms with a medical practitioner to receive one or more feedback related to the patient's health by using a communication module (shown in FIG. 3) in the server 114. Subsequently, the medical practitioner shares their feedback through the smartphone 106, and the diagnosis is provided to the operator of the base ECG unit 104 through the display of the smartphone 106.

In an embodiment, one or more features such as power, display and computational capabilities can be sourced from or through the smartphone 106. The advantage of the disclosed system is that the base ECG unit 104 is a standalone, off-the-shelf, portable device, which can connect to and use features of any smartphone 106, thereby reducing the device cost in terms of display, computational power, and various related electronics.

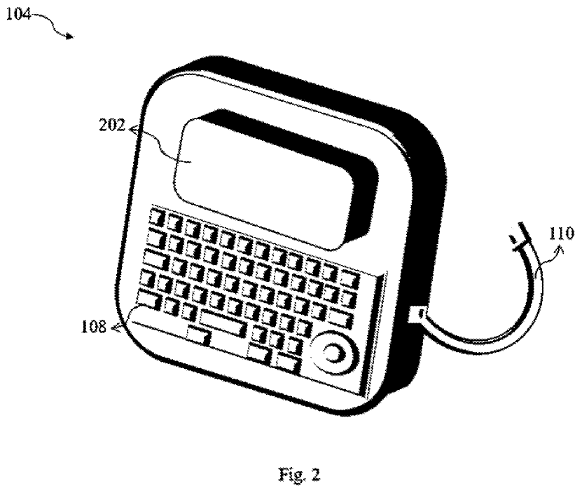

FIG. 2 depicts a base ECG unit 104, in accordance with an embodiment of the invention. One or more electronic probes 102 are connected to the patient's body to capture their ECG waveforms. The electronic probes 102 are also connected to the base ECG unit 104 to transfer the captured ECG waveforms to the base ECG unit 104.

The base ECG unit 104 comprises the keyboard 108 and ECG processing unit 122. In an embodiment, the keyboard 108 is designed with specialized keys to enter details related to the patient.

In an embodiment, an ECG processing unit 122 in the base ECG unit 104 converts the analog ECG waveforms received from the probes 102 into digital ECG waveforms.

In an embodiment, the system 100 also provides one or more features comprising compression and cleanup, to provide better ECG signals for display on the smartphone 106.

In an embodiment, one or more provided algorithms are adaptive to the capabilities of the smartphone's memory and computational capabilities. As an example, lower end smartphones 106 may include less advanced displays. Hence, an image or ECG signal may be subsampled to reduce image rendering computations, thereby improving responsiveness.

In an embodiment, in case of higher end phones, the image quality may be improved by full fidelity rendering. Additionally, the length of one or more filters and the computational load may also be scaled similarly.

In an embodiment, a GPS unit (not shown in figure) could be used to detect location and turn on filters which are geo-specific. As an example, the GPS unit can detect that the system 100 is being used in India, and thus 50 Hz notch filters made be used on the ECG signal. Similarly, 60 Hz notch filters may be used in case the GPS unit detects that the system 100 is being used in the USA.

FIG. 3 depicts the components of the ECG system, in accordance with an embodiment of the invention.

In an embodiment, probes 102 are any probes used for reading a patient's ECG waveforms, and are known in the art.

In an embodiment, the base ECG unit 104 comprises a smartphone stand 202, which can be used to hold or position the smartphone 106 while the base ECG unit 104 is being used to read a patient's ECG waveform. In an embodiment, the smartphone stand 202 is inclined to provide a better viewing angle to the operator of the base ECG unit 104. Further, the smartphone stand 202 can comprise a slot for securely and accurately positioning the smartphone 106 on the base ECG unit 104

In an embodiment, the smartphone 106 comprises a display 308, an ECG application 310, a memory unit 312, and a GPS unit 314. The ECG application 310 incorporated in the smartphone 106 processes the received digital ECG waveforms and transmits the processed ECG waveforms to the cloud 112.

In an embodiment, the smartphone 106 comprises an in-built GPS unit 314, which can access or collect location co-ordinates of the patient or the base ECG unit 104.

In an embodiment, the location co-ordinates can be utilized to identify optimum traffic routes to guide an ambulance/medical practitioner towards the location of the patient in case an emergency/critical condition was detected in the processed ECG waveforms.

In an embodiment, one or more algorithms are incorporated into the ECG application 310 to perform one or more analysis of the digital ECG waveforms. In an embodiment, the ECG application 310 can be used to perform cleaning, validation, encryption and compression of the digital ECG waveforms.

In an embodiment, the server 114 is an external server which can analyze received ECG waveforms through an ECG processing module 316 comprised within the server 114. A memory module 318 is used for storing details related to a patient, such as personal information comprising name, age, weight, address, among others. The memory module 318 can also store medical and financial information related to the patient, including one or more of financial account details, previous health reports, medical history, medical expenses, etc.

In an embodiment, the ECG base unit 104 can be used to connect to one or more devices comprising a scanner (not shown in the figure). The scanner can be used to scan an identification proof or other details that are used for maintaining patient accounts. In an example, a bar-code scanner can be used to read a patient's medical record comprising a bar code, in order to quickly access the patient's information. Thus, an added advantage of using a scanner, is that one or more of the patient's information such as their medical history, health information, patient ID, insurance information, personal information, and financial information, among others, can be accessed quickly, accurately and conveniently.

Additionally, a medical application running in the smartphone 104 can be used to interface with other blue tooth connected devices such as BP monitor/weighing machine,

In an embodiment, in case the operator is unable to operate the base ECG unit 104 accurately, one or more audio cues/information are provided to the operator through a speaker in the smartphone 106 (not shown in the figure).

In an embodiment, the smartphone 106 enables video-conferencing between the operator and the medical practitioner during an emergency, critical situation, or as required by the operator or patient.

In an embodiment, the smartphone 106 enables a remote medical practitioner to view and trigger an ECG capture by providing a feature for casting the display of the smartphone 106, and allowing the medical practitioner to access or control one or more information/options being displayed by the smartphone 106.

In an embodiment, the base ECG unit 104 is also configured to connect to one or more different medical devices/IoT devices (not shown in the figure), in order to provide an overall health screening of the patient.

In an embodiment, the ECG system 100 may be used m any national/regional emergency healthcare organizations.

In an embodiment, one or more algorithms may be incorporated in the smartphone 106 connected to the base ECG unit 104 in order to determine the computational capabilities of the smartphone 106. Subsequently, the algorithms are configured to select among one or more versions of the ECG application 220 to optimize a performance of the smartphone 106, considering the available resources in the smartphone 106.

In an embodiment, simpler and low-complexity filtering algorithms or sophisticated algorithms are utilized based on a type of smartphone 106 connected to the base ECG unit 104. Similarly, one or more image rendering algorithms can be adapted with respect to available resources, thereby providing optimum performance for the smartphone 106.

In an embodiment, one or more noise suppression filters (not shown in the figure) are utilized for filtering the processed ECG waveforms before displaying the same to the operator through the smartphone 106.

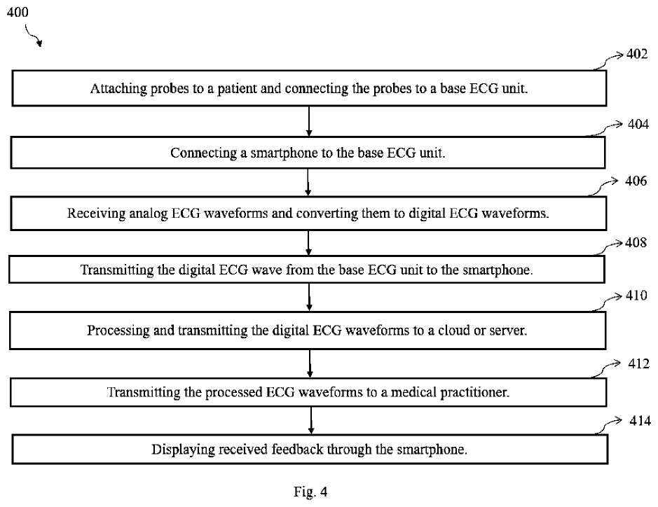

FIG. 4 depicts a flow chart illustrating a method 400 of communication between the cloud-connected base ECG unit and the medical practitioner, in accordance with an embodiment of the invention. The method 400 starts at step 402 by attaching probes to a patient and connecting the probes to a base ECG unit. At step 404, a smartphone is connected to the base ECG unit, wherein the smartphone provides power to the base ECG unit through a connection between the smartphone and the base ECG unit. At step 406, real-time analog ECG waveforms are received by the base ECG unit and converted to digital ECG waveforms. At 408, the base ECG unit transmits the digital ECG wave forms to the smartphone. Subsequently, at step 410, the digital ECG waveforms are processed and transmitted to a cloud or server. Thereafter, at 412, the processed ECG waveforms are transmitted to the medical practitioner. Subsequently, a feedback is received from the medical practitioner and is provided to the operator through a display or speaker in the smartphone.

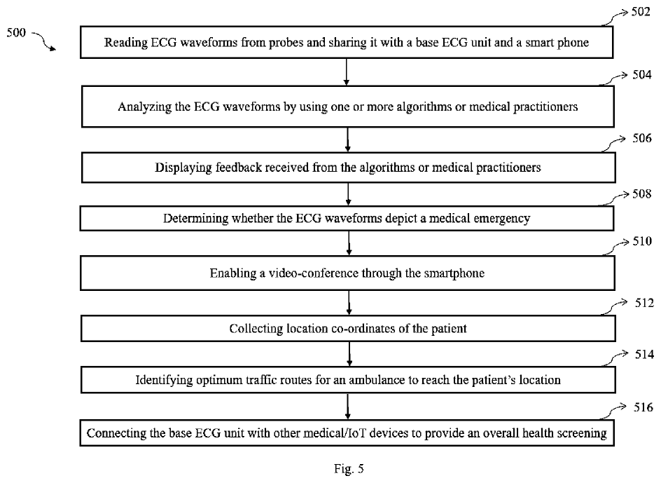

FIG. 5 depicts a flowchart illustrating a method 500 for real-time remote diagnosis, in accordance with an embodiment of the invention. The method 500 begins at step 502 by reading ECG waveforms from probes and sharing it with a base ECG unit and a smartphone 106. Further, at step 504, the ECG waveforms are analyzed by one or more algorithms or medical practitioners. At 506, feedback received from the algorithm or medical practitioner is displayed to the operator. The algorithm or medical practitioner determine whether the ECG waveforms depict a medical emergency at step 508. Subsequently, at step 510, video-conferencing is enabled between the operator and the medical practitioner through the smartphone. At 512, the GPS unit of the smartphone collects location co-ordinates of the patient. Consequently, the smartphone identifies optimum traffic routes for an ambulance to quickly reach the patient's location during the determined emergency at step 514. Further, the system is configured to connect with other medical devices/IoT devices to an overall health screening of the patient, at step 516.

The advantage of the disclosed system 100 is that the base ECG unit 104 is compact and can be easily transported. Further, the smartphone 106 can be used to power for the base ECG unit 104. Additionally, ECG waveforms are processed in the smartphone 106, due to which the base ECG unit does not comprise extra electronic circuits, which makes the base ECG unit 104 cost-effective as well as compact.

Additionally, the base ECG unit comprises a keyboard and specialized keys to enter patient details. This feature is advantageous as the specialized keys can be used to quickly access required functions and the QWERTY keyboard can be used to enter all patient details into their account. Additionally, the presence of the QWERTY keyboard allows the smartphone to be used continuously for displaying ECG data.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

1. A desktop ECG system comprising:
a base ECG unit for receiving ECG waveforms from probes attached to a patient, wherein the base ECG unit comprises:
a connection unit connecting the base ECG unit to a smartphone; and
a keyboard for entering one or more patient details;
the smartphone connected to the base ECG unit for processing ECG waveforms and communicating the processed ECG waveforms to a cloud or a server, wherein the smartphone comprises:
a display for depicting the ECG waveforms;
a memory unit for storing one or more ECG waveform data; and
an ECG application for processing and sharing one or more ECG data with a server;
the server configured to receive and process ECG waveforms, wherein the server comprises:
an ECG processing module for analyzing the received ECG waveforms; and
a communication module for sharing a feedback related to the ECG waveforms with the smartphone.

2. The system as claimed in claim 1, wherein the keyboard comprises a QWERTY keyboard for entering the patient's details, and wherein the keyboard comprises one or more specialized keys for enabling repeated functions.

3. The system as claimed in claim 1, wherein the base ECG unit is configured to connect with one or more medical devices or IoT devices to compile health data related to the patient.

4. The system as claimed in claim 1, wherein the smartphone provides power supply to the base ECG unit through a connection unit for connecting the smartphone and the base ECG unit.

5. The system as claimed in claim 1, wherein the smartphone and the base ECG unit are configured to enable video-conferencing a medical practitioner and an operator of the base ECG unit.

6. The system as claimed in claim 1, wherein the smartphone and the base ECG unit are configured to enable casting the display to a medical practitioner, and to receive instructions from the medical practitioner through the smartphone.

7. The system as claimed in claim 1, wherein one or more of the base ECG unit the smartphone and the server are configured to detect a health emergency by processing the ECG waveforms of the patient.

8. The system as claimed in claim 7, wherein location co-ordinates of the patient are collected by a GPS unit comprised within the smartphone to identify optimum traffic routes to the patient's location during a detected emergency.

9. The system as claimed in claim 1, wherein location co-ordinates of the patient are collected by a GPS comprised within the smartphone to identify one or more appropriate filters to be used on the ECG signal.

10. The system as claimed in claim 1, wherein the base ECG unit is configured to connect with one or more medical devices/IoT devices to compile health data related to a patient.

11. A method for remote monitoring of ECG waveforms by using a base ECG unit, said method comprising:
attaching probes to a patient and the base ECG unit; connecting a smartphone to the base ECG unit;
receiving real-time analog ECG waveforms through the probes;
converting the received analog ECG waveforms into digital ECG waveforms by using an ECG processing unit;
transmitting the digital ECG waveforms from the base ECG unit to the smartphone by using a connection unit;
processing the digital ECG waveforms by an ECG application m the smartphone;
sharing the processed ECG waveforms with a medical practitioner; receiving feedback from the medical practitioner; and
displaying the received feedback through the smartphone.

12. The method as claimed in claim 11, wherein patient's details are entered through a QWERTY keyboard, and wherein one or more specialized keys are used for enabling repeated functions.

13. The method as claimed in claim 11, wherein the method comprises connecting the base ECG unit with one or more medical devices or IoT devices to compile health data related to the patient.

14. The method as claimed in claim 11, wherein the method comprises providing power to the base ECG unit through a connection unit connecting the smartphone and the base ECG unit.

15. The method as claimed in claim 11, wherein the method comprises enabling video-conferencing between a medical practitioner and an operator of the base ECG unit through smartphone and the base ECG unit.

16. The method as claimed in claim 11, wherein the method comprises casting the display to a medical practitioner and receiving one or more instructions from the medical practitioner by the smartphone.

17. The method as claimed in claim 11, wherein a health emergency is detected by processing the ECG waveforms of the patient by using one or more of the base ECG unit, the smartphone, and the server.

18. The method as claimed in claim 17, wherein the method comprises collecting location co-ordinates of the patient by a GPS unit, comprised within the smartphone to identify optimum traffic routes to the patient's location during a detected emergency.

19. The method as claimed in claim 11, wherein the method comprises identifying one or more appropriate filters to be used on the ECG signal based on location co-ordinates of the patient collected by a GPS unit comprised within the smartphone.

20. The method as claimed in claim 11, wherein the base ECG unit is configured to connect with one or more medical devices/IoT devices to compile health data related to a patient.
